(19) United States
(12) Patent Application Publication
(10) Pub. No.: US 2018/0196778 A1
(43) Pub. Date: Jul. 12, 2018

(54) Method for Correlating Physical and Chemical Measurement Data Sets to Predict Physical and Chemical Properties

(71) Applicant: The University of Wyoming Research Corporation d/b/a Western Research Institute, Laramie, WY (US)

(72) Inventors: Ronald R. GLASER, Laramie, WY (US); Thomas F. TURNER, Laramie, WY (US); Jean-Pascal PLANCHE, Laramie, WY (US)

(21) Appl. No.: 15/742,203

(22) PCT Filed: Jul. 6, 2016
(86) PCT No.: PCT/US2016/041182

§ 371 Date: Jan. 5, 2018

Related U.S. Application Data
(60) Provisional application No. 62/189,110, filed on Jul. 6, 2015.

(51) Int. Cl. G06F 17/15; G06F 17/18; G01N 33/28

(52) U.S. Cl. CPC G06F 17/156; G01N 33/2823; G06F 17/18

(57) ABSTRACT

The present invention is generally related to the correlation of physical and/or chemical measurements with other physical and/or chemical measurements and the application of the correlation to transform a product or process (e.g., to formulate, mix, blend compounds or materials of various natures and origins) upon predicting/estimating certain property(ies) and/or performance index(ices) as indicated by a dependent variable estimate. Embodiments of the inventive technology applies specifically to the problem of producing a correlation when the independent variables of interest exceed the number of observations. This situation is common in many fields of science and technology, such as, but not limited to, spectroscopy, calorimetry, thermogravimetric, chromatography and others. A perhaps primary advantage of embodiments of the inventive method over prior art is the ability to generate correlations directly in terms of measured variables.

### CROSS-REFERENCE TO RELATED APPLICATIONS

This international patent application claims priority to and the benefit of U.S. Provisional Application 62/189,110, filed Jul. 6, 2015, said provisional application incorporated herein in its entirety.

### STATEMENT REGARDING FEDERAL RIGHTS

This invention was made with government support under contract DTFH61-07-D-00005 awarded by the U.S. Department of Transportation. The government has certain rights in the invention.

### TECHNICAL FIELD

The inventive technology disclosed herein is especially useful where insufficient observations are available compared to the number of independent measurement variables available. This situation is common in many fields of science and technology, such as spectroscopy, calorimetry, thermogravimetric, chromatography, and others.

### BACKGROUND ART

Modern analytical techniques often rapidly produce quite large data sets, the most common are those data sets generated using a spectrometer and are usually described as “spectra”. However, observations can be made using a wide variety of instruments/methods to generate different data; often one observation using a single instrument or can generate many pieces of data (e.g., a single IR spectrometer observation can generate 4000 absorbance data for 4000 different wave numbers). Any set of data that can be formulated as a response as a function of an index (time, wave number, temperature, etc.) can be treated as a “spectrum”, although in some scientific parlance that term is reserved for spectrometer generated data.

Consequently, a thorough examination of the relationships between a given spectrum (or other arbitrary data matrix) type and an independently measured material property can generally expressed by the following general relationship:

y=f(x₀,x₁,x₂ . . . xₙ) (1)

Where:
y is the dependent variable, e.g. complex modulus (as but one of many examples, including generally, but not limited to, either chemical or physical properties; and durability from properties measured at various aging stages, unaged and aged; see additional discussion below)
xᵢ is the independent variable(s), e.g. IR absorbance at wave number I (when the measuring instrument is, e.g., an IR spectrometer)

If f(x) is assumed to be algebraically linear, and we have only 3 spectra representing 3 materials or conditions (a,b,c, such as a first asphalt, a second asphalt, and a third asphalt; a first crude oil, a second crude oil and a third crude oil, as but two of many different examples, or three different temperatures or other conditions), along with their dependent properties of interest, the equation set is:

yₐ=k₀xₐ₀+k₁xₐ₁+k₂xₐ₂+ . . . +kₙxₐₙ (2)

y_{b}=k₀x_{b0}+k₁x_{b1}+k₂x_{b2}+ . . . +kₙx_{bn} (3)

y_{c}=k₀x_{c0}+k₁x_{c1}+k₂x_{c2}+ . . . +kₙx_{cn} (4)

where k is the proportionality constant for each wave number (or for each oil fraction, e.g.) 0 through n.

Since this is a curve fitting problem, the x and y pairs are known, and we seek k's that satisfy the equation set. Such a deterministic solution is impossible if n+1 exceeds the number of observations. When the number of observations is exactly equal to n+1, then the fit is perfect, meaning no statistical evaluation of the fit quality is possible. This is analogous to the situation in two dimensions where you are fitting a line to 2 data points, obtaining a correlation coefficient of 1. To obtain a statistically meaningful test of a multidimensional fit, the observations should exceed the independent variable count by some factor, the larger the better, but generally conceded to be a multiplier of 7. Typical mid-infrared spectra will contain nearly 4000 wave numbers, so the examination of each and every wave number for significance when combined with the others would require 28000 measurements, clearly not practical. This situation is a recurring problem with spectral data and other extensive xy data sets as well, as the inclusion of all of the data results in an equation system with excessive adjustable parameters that is impossible to solve. A number of approaches exist for addressing this problem with a variety of strategies aimed at essentially reducing the number of effective k's (independent variable fit parameters) to be discovered.

The WRI chemometric software is especially useful for applications where insufficient observations are available compared to the number of independent measurement variables available. This situation is common in many fields of science and technology, such as spectroscopy, calorimetry, thermogravimetric, chromatography and others.

A few of the methods used to address the problems outlined above are briefly described in the next three sections. These methods, particularly the process of correlating spectral data to other process or property variables, have been used successfully in a wide range of applications, but do not produce a closed form equation in terms of measured quantities, limiting their usefulness in fundamental scientific studies.

Multiple Linear Regression Multivariable regression is a time-honored technique going back to Pearson's 1901 use of it. Multivariable regression can establish that a set of independent variables explains a proportion of the variance in a dependent variable at a significant level (through a significance test of R²), and can establish the relative predictive importance of the independent variables (by comparing beta weights). Variable transformations (most common is the logarithm) can be applied to independent or dependent variables to explore some curvilinear effects, and polynomials can be fit as well by expanding independent variables into a power series.

Multivariable linear regression can solve the matrix Y=MX+B, provided sufficient measurements of Y exist to obtain all of the coefficients in vector M. To be statistically meaningful, measurements of Y in excess of measurements of X must be available, meaning that a spectra of 3500 wave numbers would require at least 3500 measurements of, say, complex modulus. To be statistically reliable, 35000 would be better. It is generally impossible to apply multivariable linear regression directly to correlation studies involving data rich spectral data. However, the preconditioning of individual data points to related groups (spectral peaks, for example) is helpful to reduce the independent variable count. However, this is usually not sufficient unless a very extensive data set (many observations) is available. A variety of computation approaches have been developed in recent years that address this problem by projecting the data in one way or another into a smaller list of independent variables. These include Principle Component Analysis (PCA), Partial least Squares (PLS), and others.

Principle Component Analysis and Principle Component Regression Principle Component Analysis techniques are applied to the problem of too many x measurements relative to y measurements by searching for so-called latent variables. The covariance of XX′ is examined and parameter space axis rotations are employed to arrive at new coordinates based on eigenvectors of the XX′ matrix. In simple terms this means that independent variables that appear to change in a similar fashion are grouped. The translated x variables (often called indicator variables) are projected into a smaller parameter space of latent variables. It is implicitly assumed that these fictitious latent variables somehow describe a truer “latent structure” to the system. Recall that the underlying mathematical model for the entire data set is linear, often patently untrue in chemical systems. This technique results in latent variable data sets with improved variance in the hopes of improving signal to noise ratios. Often, however, irrelevant data included in the translations pass spurious noise to the latent variables.

Principle components regression (PCR) is the application of ordinary linear regression methods to the latent variables developed form the principle components analysis. The difficulty with this method is that the complex axis rotations make understanding what the latent variables represent in terms of measurable quantities difficult. Interpretation of the results in terms of chemistry and physics is difficult and requires sensitivity testing by varying the input data. While useful for calibration within the testing range of the data employed, using this method for understanding the underlying science is difficult.

Partial Least Squares PCR is based on the spectral decomposition of XX′ to select latent variables for regression, while PLS is based on the singular value decomposition of X′Y. In practice, PLS usually fairs better than PCR since the reduction of parameter space dimensions is accomplished though comparison of the independent variables with the dependent variables. PCR, on the other hand, focuses mainly on what can be thought as the signal strengths of the independent variables alone for parameter space reduction, and is therefore more prone to the introduction of irrelevant signals into the regression. As with PCR, PLS suffers from the difficulty that the complex axis rotations make understanding what the latent variables represent in terms of chemistry and physics difficult and requires sensitivity testing by varying the input data. While useful for calibration within the testing range of the data employed, using this method for understanding the underlying science is difficult.

Other Methods Many other algorithms have been developed in recent years, including neural networks and artificial intelligence. While these “black box methods” can work extremely well over the calibration range used, we still are faced with the difficulty of understanding how the input variables relate directly to dependent variable without sensitivity testing.

Because of the difficulty of latent variable methods to demonstrate the correlation in terms of directly measured variables, we developed our own methods to address the issues associated with impossible and/or unfavorable parameter to observation ratios. In the simplest of terms, two strategies can be employed to make the problem tractable; reduce the independent variable count, or increase the number of observations. Once a statistically meaningful correlation can be computed, a method for selecting the most important independent variables must be applied to remove irrelevant signals and find those responses that significantly affect the quality of the fit. When applying this technique to infrared spectroscopy-to-rheology correlations, the independent variables are spectral wave numbers and represent vibrational modes of functional groups. Consequently, important clues about how chemical changes cause rheological changes can be obtained.

### SUMMARY OF THE INVENTION

This method provides a process to generate correlations between physical and chemical measurements, chemical and chemical measurements, and physical and physical measurements when sufficient observations are not available to perform the correlation while examining all of the measurements at once. Indeed, embodiments of the inventive “chemometric” software are especially useful for applications where insufficient observations are available compared to the number of independent measurement variables available. Unlike prior art, which reduces the independent variable count to “latent variables” through dimensional reduction accomplished by complex rotations and projects, as is the case for partial least squares and principle components analysis schemes, embodiments of the inventive method produce correlations that are expressed in closed form mathematical equations in terms of the measured values of significance.

Stepwise multivariable regression also produces correlations in measured value terms, but is unable to examine all combinations in the independent variable list at once; hence some combinations are not tested. Prior art focuses upon independent variable reduction schemes, while this method uses independent variable reduction scheme cast explicitly in terms of the measured values, and, uniquely can also expand the data set by producing additional artificial observations based upon the known (or determined or estimated) precision of the measurement methods. This expansion of the regression data set provides a key method for not only producing a “fit” of the data, but also assessing the significance of the parameters used using any of a variety of well-established statistical methods for estimating parameter significance and parameter rejection criteria.

The invention comprises using an approach employing the new chemometrics software with data from a one or more chemical and spectroscopic analysis methods to generate relationships with selected physical properties. The results of the correlations will provide equations that could be interpreted in a manner enabling an understanding of how the analysis results reflect the physical behavior. This approach can be used to evaluate current properties and to predict changes in properties following aging or treatment.

The present invention is generally related to the correlation of physical and/or chemical measurements with other physical and/or chemical measurements. This method applies specifically to the problem of producing a correlation when the independent variables of interest exceed the number of observations. The advantage to this method over prior art is the ability to generate correlations directly in terms of measured variables. The WRI chemometric software is especially useful for applications where insufficient observations are available compared to the number of independent measurement variables available. This situation is common in many fields of science and technology, such as spectroscopy, calorimetry, thermogravimetric, chromatography and others.

### BRIEF DESCRIPTION OF DRAWINGS

FIG. 1 is a flow chart of the chemometric method to obtain relationships between independent variables measured and dependent variables measured.

FIG. 2 is an example of grouping of IR spectra absorbances with absorbance at 2000 cm⁻¹.

FIG. 3 is a graph showing one example of modified automated SAR-AD separation profile of an asphalt.

FIG. 4 is a graph showing one example of size exclusion chromatography (RI detector) profiles for eight asphalts.

FIG. 5 is a graph showing one example of penetration (PEN) correlation coefficients.

### DESCRIPTION OF EMBODIMENTS OF THE INVENTIVE TECHNOLOGY

As mentioned earlier, the present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

An assigned linear dependence of a dependent variable on a plurality of independent variables may be as follows:

yₐ=k₀xₐ₀+k₁xₐ₁+k₂xₐ₂+ . . . +kₙxₐₙ (2)

y_{b}=k₀x_{b0}+k₁x_{b1}+k₂x_{b2}+ . . . +kₙx_{bn} (3)

y_{c}=k₀x_{c0}+k₁x_{c1}+k₂x_{c2}+ . . . +kₙx_{cn} (4)

where k is the proportionality constant for each wave number (if the measuring instrument is an IR spectrometer, or for, e.g., each oil fraction, (if the measuring instrument is a SAR-AD analyzer), as but two examples, 0 through n. Note that a single observation can produce “n” measurements (e.g., where the observation instrument is an IR spectrometer, perhaps 4000 measurements (“n”=4000) are made during that single observation (one for each wavelength); where the observation instrument is SAR-AD, perhaps 16 measurements are made (“n”=16).

Note also that while certain embodiments may include the step of performing “p” number of observations to obtain “p” number of measurements for each said dependent variable and said independent variables, wherein “p” is less than the sum of “n”+1, other embodiments may include the step of performing “p” number of observations to obtain “p” number of measurements for each said dependent variable and said independent variables, wherein “p” is less than “n.”

Where a relationship between a dependent variable (e.g., of a product, process, ingredient of a product, material that is acted on or used in any way in a step of a process, etc.) and a plurality “n” of independent variables is either known to be linear, suspected as linear, presumed linear (whether to test a fit or other reasons), or in any way treated as linear (whether by computer, software, operator, etc.), it is said that linear dependence of the dependent variable on “n” number of independent variables is assigned. Note that a computer that in any way treats, mathematically, e.g., via coded instructions, said relationship as linear is said to assign such linear dependence. Even where it may eventually appear that some of the independent variables do not have a linear impact on the dependent variable, that does not prevent the fact that at the initial stages of the inventive protocol disclosed herein, such linear dependence was assigned.

X Measurements (measurements of the independent variables) include but are not limited to the following, or measurements of the following phenomenon/properties, or measurements made using the following analysis/instruments:
temperature, asphaltene %, IR wave number/length, UV Absorbance;
Spectroscopy: IR, NIR, MIR wavelengths and band intensities, NMR displacement, peak intensity, UV, RAMAN, SAX, SANS and XRay diffraction . . . ;
Composition: elemental analysis, metal content;
Microscopy and image analysis, Electronic, Optical, Atomic (AFM), tomography, MRI;
Thermal properties: DSC glass transition temperature, crystallinity, TGA weight loss, HP DSC oxidation induction time;
Separation: All SAR-AD, WAX-AD, SARA fractions and indices, Automated Flocculation Titrimeter (AFT) indices, GPC molecular weight or retention times and intensities, IEC (note that more details regarding the SAR and SARA separation may be found in Boysen, Ryan and Schabron, John, Automated HPLC SAR-AD Separation; Fundamental Properties of Asphalts and Modified Asphalts III Product: FP 01, March 2015, which is incorporated herein by reference in its entirety);
Olefin index;
Acidity-Basicity: TAN, TBN;

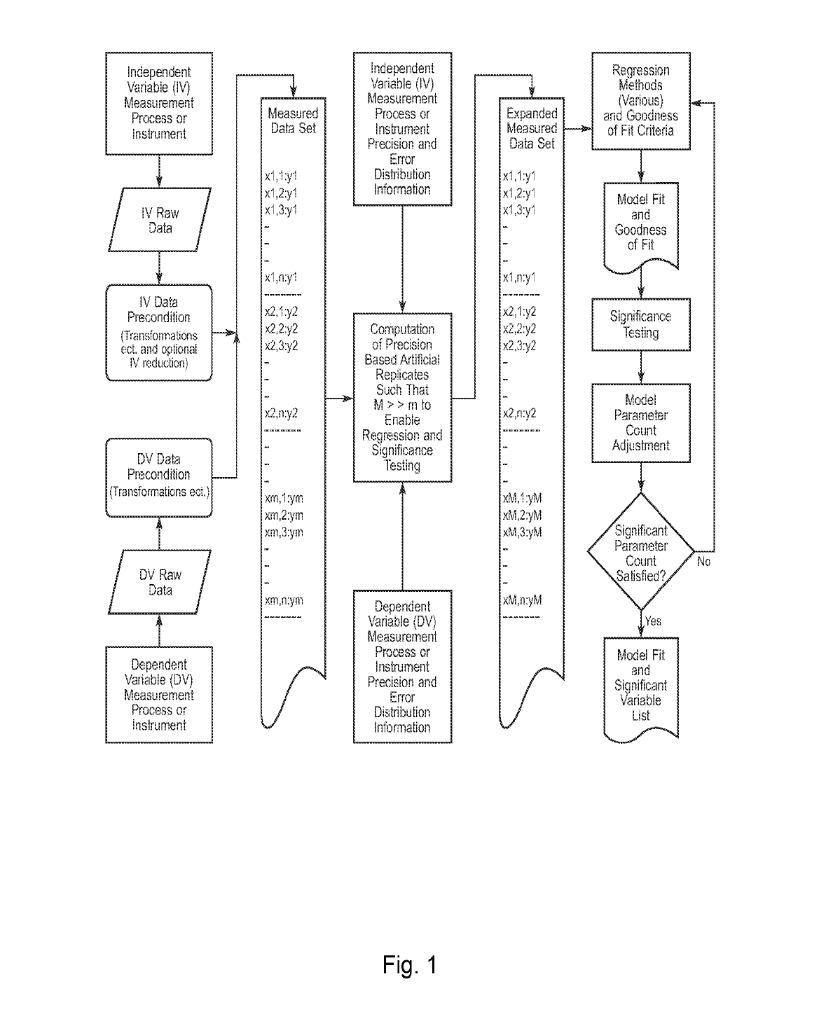

Standard procedures may be used to estimate the instrument or method measurement precision, if it is not known (e.g., as provided by the instrument manufacturer or process standardizing body such as ASTM), which may include the precision distribution, of the methods used for the collection of independent and dependent data. These measurements may be spectrographic, chromatographic, calorific, gravimetric, thermo-gravimetric, or even any measurement process that produces a numerical value. A flow chart of the chemometric method is provided in FIG. 1. Indeed, these steps may describe identically an embodiment of the inventive technology. In addition to other measuring instruments/analytical tools indicated above and elsewhere herein, measurements (particularly of chemical properties) may be obtained using, e.g., using the following analytical tools/measuring instruments alone or in combination, as examples and non-exclusively:
IR, NIR, MIR;
SAR-AD, WAD and other SARA methods;
NMR (1H and 13C);
GPC/SEC;
DSC;
IEC; and
AFT, as but a few examples.

More particularly with regard to physical properties, the following are merely examples of the many instruments/analytical tools that could be used to generate measurements of such properties: DSR, BBR, ABCD, DMA, mechanical test, and fouling apparatus, as but a few examples.

Materials that may be measured, materials for which a dependent variable may be estimated, materials that may be transformed, and materials as to which a process may be transformed using the inventive method (e.g., by estimating a dependent variable) include but are not limited to: petroleum, coal, and biomass products, fuel, medication, dietary supplements, cosmetics, food, lubricants, and any other materials indicated in this application, or in references incorporated herein. Such material(s) may certainly be related to the product or process that is transformed (indeed, such material may be that product); that material(s) may be an ingredient in a process, anyway involved in a step of the process, may be a part of the product, a material that the product is a part of, as but a few examples.

Y measurements (and indeed parameters that can be estimated upon determination of coefficients of significant independent variables) include but are not limited to the following parameters/indexes/properties, and/or parameters/indexes/properties related to the following phenomenon, and/or measurements thereof:
Any measurement related to crude oil/petroleum fouling, coking, emulsion ability, stability, instability;
Gas cetane, octane numbers;
Any measurements related to lubricants: anti-wear properties, viscosity index, oxidation resistance, fluidity, tribology;
Asphalt penetration, ring and ball softening point, fraass brittle point, viscosity, modulus, phase angle, Superpave properties, DSR, BBR critical temperatures, oxidation resistance short term and long term;
Material fatigue resistance, brittleness, hardness, elasticity, plasticity, deformation, roughness, density;
Any index related to organic, inorganic material Oxidation, weatherability, durability inflammability, explosiveness, carcinogenicity, mutagenicity;
Metal corrosion;
Liquid or paste fluidity, thixotropy, viscosity, density;
perfume smell, spraying ability.
medication efficiency/effectivenes,
Viscosity, material hardness, reflectivity

Often, but not always, X measurements are measurements of a property, phenomenon, etc. of the same material that the Y measurements relate to.

Preconditioning The raw data may be preconditioned by any number of variable transformations, ratios, normalization as deemed useful for data interpretation. Grouping/Consolidation may be viewed, in certain embodiments, as a type of preconditioning.

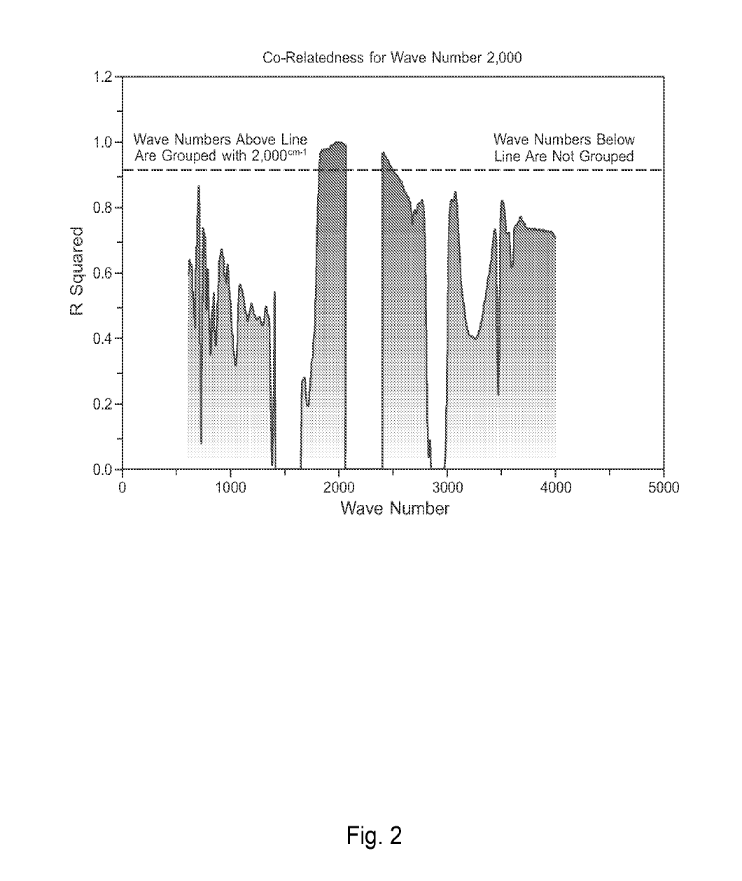

Grouping/Consolidation of Independent Variables Additionally perhaps, and possibly but not necessarily as a preconditioning step, the independent variable list count may be reduced by correlating an independent variable with one or more of the remainder of the independent variables, and consolidating/grouping, where appropriate, them into a single variable if the quality of fit of a given variable pair (or generally, grouping, if there are more than two independent variables that sufficiently correlate) exceeds a user defined value (e.g., where R² corresponding to the two or more independent variables is above a certain value). An example of this is provided in FIG. 2. The consolidation of several independent variables may be based upon any of a number of criteria, such as best signal to noise ratio, averages, weighted averages, geometric means, or other formulations best suited to the type of measurement involved. Note also that the term independent variables includes even those variables that, after analysis (e.g., grouping analysis), are found not to be entirely independent of one another (e.g., when one increases, another increases in linear relation).

Grouping combines independent variables together if they contain the same information. This reduces the number of independent variables without reducing information content. Grouping may find particular application to spectra where there are thousands of data points, many of which are most likely not relevant. F-Test (Add/Reject) may remove the least significant variables, i.e., those with no statistically relevant information. Grouping may be a type of preconditioning. In certain embodiments, if grouping does not reduce the number of independent variables enough, then generation of artificial replicates may be required.

Of course, the highest quality data is desired. Grouping (like artificial replication) cannot improve data quality; instead, its main benefit may be speeding the analyses. At present, large datasets require days to analyze using a computer (and indeed using the inventive software where independent variables have not been sufficiently reduced), and days for us to evaluate the results, and more days to write a report. Without grouping, at times, weeks could be required to obtain results; with grouping (and perhaps other steps such as independent variable reduction using, e.g., F-Test (Add/Reject); with grouping and perhaps other data reduction, results can be obtained much faster.

Artificial Replication In order to make the correlation mathematically tractable, the number of observations must exceed the number of independent variables by one. Statistical validity requires a larger number, generally considered to be at least seven times the number required to solve the equation matrix. For simple systems, one can design the experiment or measurement program to collect sufficient observations, but often this is not practical. By knowing the precision of the measurement method (e.g., of the instrument used to measure, and/or the method or protocol (e.g., ASTM method) used), and the distribution of the precision error (collectively these may be referred to as measurement precision), a set of artificial measurements can be generated that looks very much like the set that would be created through physical measurement. These artificial replicates are created by averaging physically produced replicates to get the best mean value, if available, and then each independent variable measurement is used as a basis for creating an additional artificial replicate using “measurement precision”, which, in particular embodiments, suggests adding or subtracting a randomly generated value within the range of precision of the measurement at a frequency determined by the (known or estimated) distribution that is characteristic of the particular measurement type used. Note that in some embodiments, one example of accomplishing the step of replication of artificial data at a measurement instrument's/measuring method's frequency may be conceptualized as selecting an unmeasured value from a series of concentric circles or annular rings centered on a measured value (or a mean/median, etc. of several measured values), each circle or ring corresponding to a range of values (e.g., uppermost and lowermost, diametrically opposed portions of one ring may correspond to a range from 17.05 to and including 17.23) and to a respective probability (e.g., a smallest radius ring may have a probability of 15%, a next largest (perhaps having an identical radial width) may have a probability of 9%, the next largest ring (perhaps having an identical radial width) may have a probability of 3%, etc.); whenever this conceptual model suggests datum generation within a certain ring, a random number generator may be used to generate a number within the range represented by that ring. Accordingly, in some embodiments, replicating artificial data using measurement precision may involve the steps of adding a number selected from certain appropriate ranges at frequencies corresponding to those ranges (for example, in the 17.05-17.23 range example given above, if a measurement within this range is expected to occur 5% of the time, then a random number between 0.01 and 0.18 may be selected and then added to 17.05 for 5% of the artificially generated measurements). However, this is merely one example of many different ways in which random numbers and a frequency profile could be used to artificially replicate data. The replication of artificial data process may continue until sufficient artificial replicates are generated to expand the experimental matrix to a size suitable for statistical study and mathematical tractability. Artificial replication, like grouping, cannot improve data quality, but can make an analysis possible. Typically, measurement precision of both the independent variables and the dependent variables is of concern and is considered.

Model Fitting Any form of regression, or curve fitting can be employed with the expanded data set. Any number of linear and non-linear algorithms may be employed.

Determining Statistically Significant Variables/Parameter Reduction A number of parameter reduction criteria exist in the prior art, including p test, F test, rate of decline of goodness of fit, and others. These often require multiple fits to compare the correlation quality with and without the particular independent variable being tested. So, parameter reduction to determine the important factors in the phenomena under study usually involves repeated fitting and quality of fit comparisons, with less significant variables being rejected one by one. Independent variables that have a statistically insignificant impact on the dependent variable can be ignored without having a statistically significant impact on results (or without impairing results to an unacceptable degree), but those having a statistically significant impact on the dependent variable are considered, and coefficients for them are later determined (coefficients for statistically insignificant independent variables may be set to zero). This differs from stepwise regression in that all variables are correlated initially, and removed one by one, rather than being added to the model one at a time. Rejection criteria can vary and often require the judgment of the investigator. After it is determined which independent variables are statistically significant (or which are not, which could yield similar or identical information), the independent variable count may be adjusted.

The end result of this method is a series of models with decreasing parameter counts and quality of fit metrics, all in terms of measured quantities. The advantage to this over prior art is particularly acute in fundamental research where causality can be studied with further testing. Note that grouping of independent variables is not always required to obtain a soluble equation set, and this method does require grouping in all cases. In addition, prior art approaches to independent variable reduction, such as Principle Components analysis, Partial least Squares, Neural Networks or Artificial Intelligence and others can also be used to reduce the independent variable count and discover significant measurements to apply to the multivariable regression step.

Upon determining coefficients for each of the statistically significant independent variables, a closed form mathematical relation may be developed (it may have fewer than n independent variables, each represented by “x”, coefficients for each of such variables, and a dependent variable. This relationship may be truncated (in other words, abbreviated or shortened) in that it has fewer than “n” independent variables (because it may only include statistically significant variables (whether they be consolidated/grouped or not)). Accordingly, particularly as compared with large data sets (with “n” total measurements), results may be generated more quickly, even where the relationship (and perhaps the entire inventive protocol) are computer implemented. The truncated relationship may be used to generate estimates of the dependent variable upon input of measurements (e.g., as numerical data) of statistically significant independent variables. That estimate can then be used to transform a process or product from what that process or product would be without consideration of that dependent variable estimate. Such transformation of process or product may be as described in more detail elsewhere in this disclosure. Because it may be known that a certain dependent variable value (e.g., within a certain range) suggests that a certain step be taken or acts be taken to achieve a certain benefit (e.g., such as using a particular additive, adding ingredients in a certain ratio, heating to a certain temperature, as but a few examples) to achieve a desired benefit (e.g., improved wearability, resistance to UV induced fading, coking risk mitigation, etc.), the dependent variable estimate (e.g., achieved using the truncated relationship) can be used to modify a process or product to achieve an improvement in that process or product.

Example of Correlations with Physical Properties:

A brief description of the use of the correlation method which is the subject of this invention is provided below for eight unaged asphalt binders. For this example, results of binder penetration (PEN) tests were correlated with results from several analysis techniques which are described below. Many other chemical and spectroscopic analyses and many other physical properties can be correlated in this manner. Some results from the analyses provided good correlations, and others did not.

Fourier Transform Infrared (FTIR) Spectroscopy FTIR spectra were obtained using an Agilent Cary 630 FTIR spectrometer to conduct analyses for this project. Solutions were 1.2 weight percent asphalt binder in tetrachloroethylene. Absolute peak absorbance values were used for the correlations.

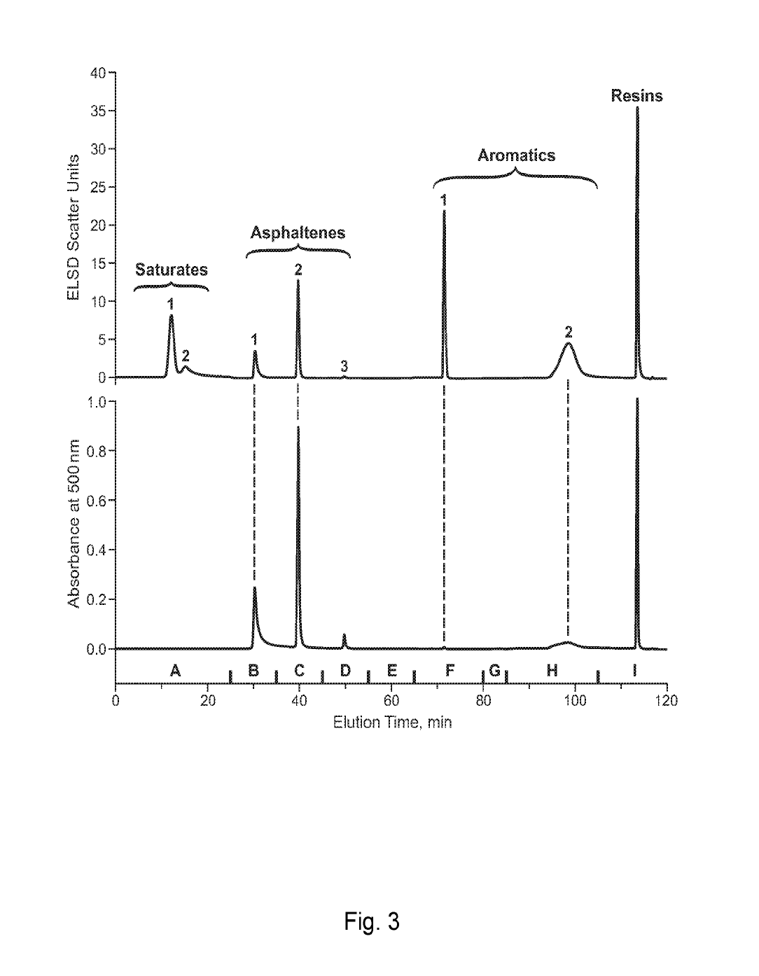

Saturates, Aromatics, Resins-Asphaltene Determinator (SAR-AD™) Separation The automated SAR separation coupled with automated AD separation (SAR-AD) is described by Boysen and Schabron (2013). The combined system, SAR-AD, generates saturates, aromatics, and resins (SAR) chromatographic fractions and elutes cyclohexane soluble, toluene soluble, and methylene chloride-methanol soluble asphaltene subfractions. The separation couples a high performance liquid chromatography (HPLC) based SAR separation with a previously-developed asphaltenes analysis method called the Asphaltene Determinator® (Schabron et al. 2010) which characterizes asphaltenes by solubility. One observation using the SAR-AD may yield several (e.g., 16) measurements, each corresponding perhaps to a single different fraction. The separation was further modified to separate the resins fraction into two fractions. Solutions of asphalt were prepared as 10% (wt/vol) in chlorobenzene. The solutions were filtered through 0.45 micron syringe filters into autosampler vials. Portions of 20 μL were injected for the SAR-AD separation. All separation profiles were electronically blank subtracted prior to peak integration. A representative SAR-AD separation profile is given in FIG. 3.

Peak Descriptions (from Left to Right on an ELSD Separation Profile (See FIG. 3))

Peak 1. Saturates: Elutes through all four columns with heptane, fully saturated alkyl molecules (model compound cholestane is in this fraction),

Peak 2. Naphthene Saturates: Elutes through all four columns with heptane, but the elution time is retarded by the activated silica. This material absorbs some light at 230 nm and 260 nm and very little at 290 and 310 nm indicating this material may contain some hydrocarbons with one or two aromatic ring structures with significant amounts of alkyl side chains.

Peak 3. Cyclohexane Soluble Asphaltenes: Highly alkyl substituted, polar, pericondensed aromatics

Peak 4. Toluene Soluble Asphaltenes: Polar, more pericondensed aromatics

Peak 5. CH₂Cl₂:MeOH Soluble Asphaltenes: Pre-coke, polar, most pericondensed aromatics

Peaks 6 and 7 Combined. Aromatics: Total aromatics. The cut between these peaks is very sensitive to the activity of the aminopropyl bonded silica, which can change with temperature humidity, and solvent purity. These peaks are combined to increase precision in the total aromatics fraction.

Peak 8. Resins: Polar heptane soluble material that elutes with CH₂Cl₂:MeOH (98:2 v:v) from the amino-propyl bonded silica and glass bead columns; some of this material absorbs visible light at 500 nm

Calculated Parameters

Coking Index: Ratio of peak areas of cyclohexane soluble asphaltenes to CH₂Cl₂:MeOH soluble asphaltenes which is a measure of pyrolysis severity history. Values below 1.0 for 500 nm peak areas indicate the presence of coke.

Asphalt Aging Index: Ratio of the toluene soluble asphaltenes 500 nm peak area to the sum the resins and aromatics fractions 500 nm peak areas. Absorbance at 500 nm is due to the presence of extended pi systems that impart brown color to oil, which increase with oxidation.

Total Pericondensed Aromatics (TPA): The approximate weight percent of material in the sample that absorbs 500 nm (visible) light.

Elemental and Metals Analyses Table 1 contains the elemental and metals results for eight asphalt samples. CHNOS analyses were performed on the neat asphalts by Huffman Laboratories, Golden, Colo. Metals analyses at Huffman Laboratories were performed on the 10% nitric acid solutions from the wet ash/dry ash procedure performed at WRI. A quality control sample was submitted with the metals solutions. The results indicated that the sample prep and analysis were in control.

TABLE 1
Elemental and Metals Analyses Results.

| | Asphalt Binder | | | | | | | |
|---|---|---|---|---|---|---|---|---|
| Analysis | B1 | B2 | B3 | B4 | B5 | B6 | B7 | B8 |
| weight % | | | | | | | | |
| Carbon | 84.27 | 84.74 | 83.10 | 83.21 | 83.75 | 83.99 | 85.24 | 84.28 |
| Hydrogen | 9.94 | 9.98 | 9.86 | 9.60 | 9.93 | 10.21 | 10.24 | 10.08 |
| Nitrogen | 0.68 | 0.45 | 0.44 | 0.45 | 0.58 | 0.51 | 0.50 | 0.43 |
| Oxygen | 0.64 | 0.56 | 0.44 | 0.43 | 0.53 | 0.60 | 0.50 | 0.50 |
| Sulfur | 4.45 | 4.59 | 6.30 | 6.30 | 5.02 | 4.62 | 3.64 | 4.28 |
| Total | 99.98 | 100.32 | 100.14 | 99.99 | 99.81 | 99.93 | 100.12 | 99.57 |
| H/C ratio | 1.41 | 1.40 | 1.41 | 1.37 | 1.41 | 1.45 | 1.43 | 1.43 |
| μg/g | | | | | | | | |
| (ppm) | | | | | | | | |
| Copper | <0.35 | <0.35 | <0.35 | <0.35 | <0.35 | <0.35 | <0.35 | <0.35 |
| Iron | 27.8 | 31.4 | 16.9 | 17.6 | 28.4 | 23.6 | 35.3 | 35.9 |
| Nickel | 107 | 35.8 | 36.1 | 38.9 | 50.0 | 84.6 | 45.6 | 36.2 |
| Vanadium | 413 | 115 | 129 | 144 | 174 | 251 | 141 | 115 |
| V/Ni ratio | 3.86 | 3.21 | 3.57 | 3.70 | 3.48 | 2.97 | 3.09 | 3.18 |

Automated Flocculation Titrimetry (AFT) The titration method is described in ASTM D6703, which was developed at WRI. Titrations were conducted using toluene solutions of asphalt titrated with heptane using an automated system. Calculated parameters include the state of peptization, P, which has a theoretical lower limit if 1.0 (highly instable), however values of P commonly vary between 2.5 to 10 for unmodified or neat asphalts. Low P values indicate internally incompatible material. Values in P are calculated as a function of two parameters that relate to the peptizability of the asphaltenes pₐ and the solvent power of the maltenes, p). The AFT data are listed in Table 2.

TABLE 2
Automated Flocculation Titration Calculated Parameters for
Eight Asphalts.

| | |
|---|---|
| AFT | Trial |

| Binder | Parameter | 1^{st} | 2^{nd} |
|---|---|---|---|
| B1 | pₐ | 0.629 | 0.631 |
| | pₒ | 1.03 | 1.01 |
| | P | 2.76 | 2.73 |
| B2 | pₐ | 0.691 | 0.694 |
| | pₒ | 1.05 | 0.98 |
| | P | 3.41 | 3.22 |
| B3 | pₐ | 0.714 | 0.710 |
| | pₒ | 1.02 | 1.21 |
| | P | 3.56 | 4.18 |
| B4 | pₐ | 0.715 | 0.716 |
| | pₒ | 1.12 | 1.05 |
| | P | 3.93 | 3.70 |
| B5 | pₐ | 0.665 | 0.660 |
| | pₒ | 1.22 | 1.30 |
| | P | 3.65 | 3.83 |
| B6 | pₐ | 0.655 | 0.662 |
| | pₒ | 1.14 | 1.04 |
| | P | 3.29 | 3.07 |
| B7 | pₐ | 0.702 | 0.695 |
| | pₒ | 0.88 | 1.00 |
| | P | 2.96 | 3.27 |
| B8 | pₐ | 0.693 | — |
| | pₒ | 1.05 | — |
| | P | 3.43 | — |

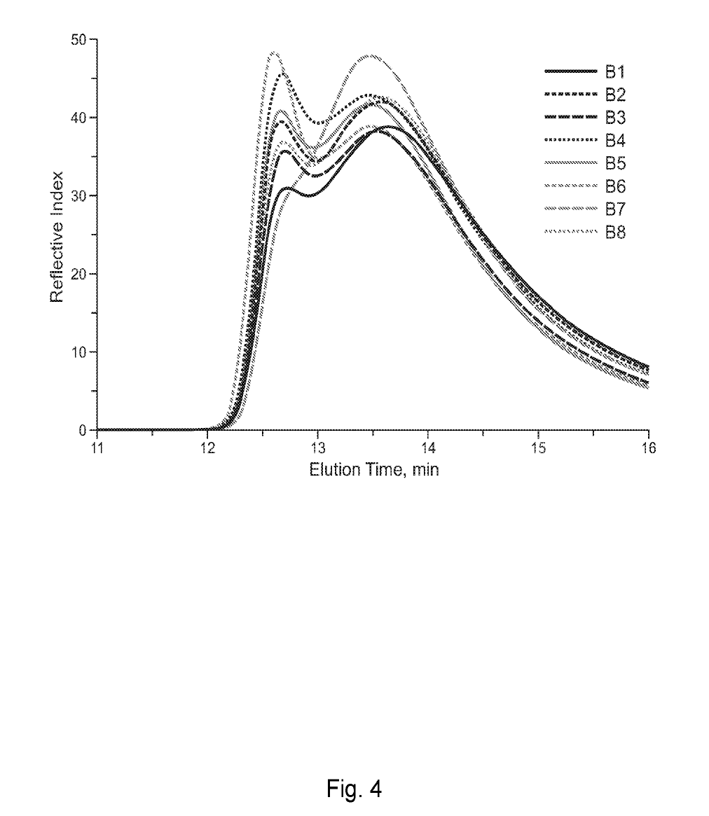

Size Exclusion Chromatography (SEC) Sample solutions were prepared by dissolving 0.30±0.0005 g in tetrahydrofuran (THF) and bringing to volume in 10 mL volumetric flasks to generate 3 wt/vol % solutions. Solutions were filtered through 0.45 μm syringe filters and 30 μL aliquots were injected into a high performance liquid chromatography (HPLC) system equipped with a 7.8×300 mm, 5 μm, 50 Å Phenogel column thermostatted to 35° C. and THF eluent at 0.5 mL/min. The THF was HPLC grade stabilized with butylated hydroxyltoluene (BHT). A differential refractive index (RI) detector was used to record the separation profiles. A second order curve fit of polystyrene standards of peak molecular weights (MW) 3000, 1300, 890 and 370 Da (g/mol) respectively was used to calibrate the system. Chromatograms were split into slices for analysis consisting of material>2966 Da, material<2966 and >1000 Da, material<1000 and >700 Da, material<700 and >370 Da, and material<370 Da. It is very likely that material<400 Da does not exist within asphalt and detection of material this size is likely due to reversible adsorption effects by polar type compounds on the column. FIG. 4 shows the RI profiles for the eight binders, and Table 3 summarizes the data.

TABLE 3
Size Exclusion Chromatography Data Summary.

| | | Slice 1 | Slice 2 | Slice 3 | Slice 4 | Slice 5 |
|---|---|---|---|---|---|---|
| Binder | Parameter | >3050 | 3050-1000 | 1000-700 | 700-370 | <370 |
| B1 | Mₙ (Da) | 3513 | 1554 | 836 | 513 | 280 |
| | M_{w} (Da) | 3577 | 1715 | 845 | 531 | 287 |
| | % in Peak | 9.06 | 41.12 | 15.52 | 21.67 | 12.63 |
| B2 | Mₙ (Da) | 3541 | 1575 | 837 | 514 | 280 |
| | M_{w} (Da) | 3608 | 1741 | 846 | 532 | 287 |
| | % in Peak | 11.55 | 42.74 | 14.66 | 19.66 | 11.39 |
| B3 | Mₙ (Da) | 3495 | 1580 | 837 | 515 | 281 |
| | M_{w} (Da) | 3555 | 1747 | 847 | 534 | 288 |
| | % in Peak | 10.75 | 44.61 | 14.93 | 19.32 | 10.4 |
| B4 | Mₙ (Da) | 3523 | 1599 | 837 | 514 | 281 |
| | M_{w} (Da) | 3585 | 1770 | 846 | 533 | 288 |
| | % in Peak | 12.52 | 44.33 | 14.17 | 18.69 | 10.28 |
| B5 | Mₙ (Da) | 3539 | 1588 | 839 | 518 | 282 |
| | M_{w} (Da) | 3603 | 1755 | 848 | 537 | 289 |
| | % in Peak | 12.89 | 46.51 | 14.48 | 17.43 | 8.69 |
| B6 | Mₙ (Da) | 3606 | 1601 | 838 | 516 | 281 |
| | M_{w} (Da) | 3682 | 1774 | 847 | 534 | 288 |
| | % in Peak | 16.64 | 43.42 | 13.6 | 17.13 | 9.2 |
| B7 | Mₙ (Da) | 3457 | 1547 | 839 | 516 | 281 |
| | M_{w} (Da) | 3510 | 1697 | 848 | 535 | 288 |
| | % in Peak | 7.15 | 47.22 | 15.68 | 19.39 | 10.55 |
| B8 | Mₙ (Da) | 3519 | 1564 | 837 | 514 | 280 |
| | M_{w} (Da) | 3582 | 1727 | 846 | 533 | 288 |
| | % in Peak | 10.4 | 42.99 | 15.13 | 20.11 | 11.37 |

Multiple Linear Regressions The correlations were evaluated by WRI's advanced multiple linear regression software. This program was designed to investigate relationships between independent and dependent variables using standard multivariable linear regression algorithms, but with some added features. In addition to solving classical multivariable data fitting problems, additional methods are available for fitting data sets with unfavorable dependent-to-independent variable ratios. This method provides a process to generate correlations between physical and chemical measurements, chemical and chemical measurements, and physical and physical measurements when sufficient observations are not available to perform the correlations while examining all of the measurements at once. An example of SAR-AD correlation parameters that may be used is provided in Table 4.

TABLE 4
Example of SAR-AD ® Correlation Parameters.

| SAR-AD Fraction | ELSD | 500 nm Absorption |
|---|---|---|
| Total Saturates | x | |
| Total Aromatics | x | X |
| Resins | x | X |
| Cyclohexane Asphaltenes | x | X |
| Toluene Asphaltenes | x | X |
| CH₂Cl₂/MeOH Asphaltenes | x | X |

Calculated Metrics

ELSD Toluene Asphaltenes to Aromatics Ratio
500 nm Toluene Asphaltenes to Aromatics Ratio
ELSD Resins to Aromatics Ratio
ELSD Resins to Total Asphaltenes Ratio
ELSD Total Asphaltenes
ELSD Resins plus Total Asphaltenes
ELSD Total Saturates plus Total Aromatics
Total Pericondensed Aromatics (TPA)
The weight percent of material in the sample that absorbs 500 nm
500 nm Coking Index
Ratio of 500 nm Cyclohexane Asphaltenes to 500 nm
CH₂Cl₂:MeOH Asphaltenes. Values below 1.0 for 500 nm peak
areas indicate presence of coke.
ELSD Coking Index
Ratio of ELSD Cyclohexane Asphaltenes to ELSD CH₂Cl₂:MeOH
Asphaltenes.
Absorbance Aging Index
Ratio of 500 nm Toluene Asphaltenes to 500 nm Resins.

Unlike most multiple linear regression programs, which reduce the independent variable count to “latent variables” through dimensional reduction by complex rotations and projections (as is the case for partial least squares and principle components analysis schemes), this method produces correlations that are expressed in closed form mathematical equations in terms of the significant measured values.

The correlation software algorithm uses two test methods to reject or accept parameters in a model. The F-Test Reject method fits data using all independent variables and then removes the least significant variable based on the F-test. This cycle of fitting and rejecting parameters is repeated until only one parameter is remaining. The R² values for all the fits are plotted versus the number of parameters to produce an “Over fit” plot. This plot provides a visual clue for how many parameters are relevant for a given fit. Having too many parameters results in over fitting, where the model is meaningless.

The F-Test Reject method, because it rejects variables one at a time, sometimes rejects independent variables that, in combination, would produce acceptable models. The F-Test Add method was developed to approach the fitting process from the other end and to reduce the chances of missing important correlations. In this method, the fitting starts with a fixed set of independent variables (usually selected from the best single variable fits) and increases the independent variable count one at a time based on the significance of the added variable to the model using the F-test.

Parameter Transformations Several parameter transformations were used to aid in the search for relevant correlations. Table 2 summarizes the whole correlation effort and shows the algebraic forms of the most common transformations used. All logarithmic transformations used natural logarithms.

Example of Correlation Results Results for the eight unaged asphalt binders were examined for this example. These represent a small sample set and interpretations and applications of the correlations should be used with care. A three or four metric correlation may be meaningful or not.

The WRI multivariate regression software (MLS) was used to look for correlations with penetrometer test data for these binders. In MLS, all independent variables are used at the start. The variables are removed one by one based on how much they influence the overall fit. Caution must be used in properly grouping the independent variables to obtain the most meaningful relationships.

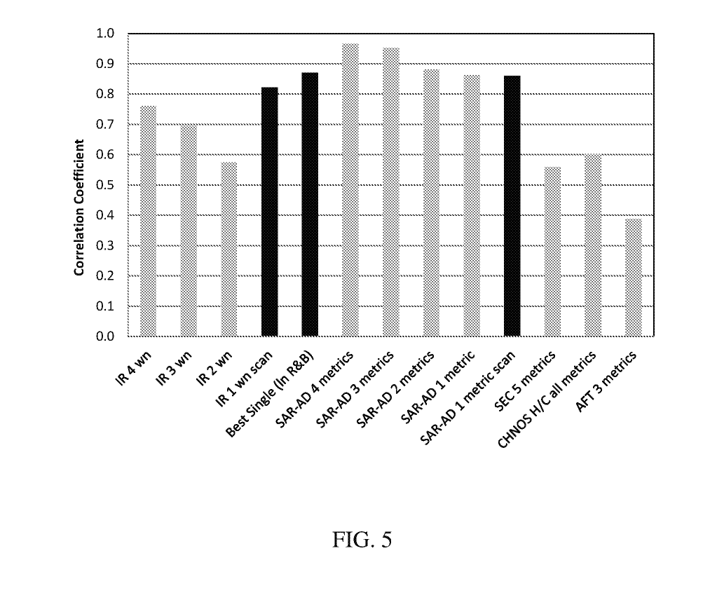

Correlations for the penetration data are shown in FIG. 5. The best single variable correlations, colored black in FIG. 5, are the following:
IR absorption at 3754 cm⁻¹ with an R² of 0.82, and
SAR-AD (Nap ELS) with an R² of 0.86.

The best multivariable correlations are also shown in FIG. 5 with the gray bars.

### APPLICATIONS OF THE INVENTION

Embodiments of the present invention generally relate to the use of the specialized WRI chemometric software for the determination of mathematical relationships between physical and chemical measurements. The invention may be used to estimate numerical values for coefficients (e.g., linear) for each statistically significant independent variable to generate a closed form mathematical equation which can be used to predict/estimate a dependent variable based on knowledge (e.g., upon measurement) of only such statistically significant independent variables. Ease, simplicity, and in many cases, speed of results, without impairing results to an unacceptable degree, may be particularly valuable benefits of the inventive technology, which often may be embodied in a computer program or software, and applied to a particular problem upon user input of measured data. Another may be, at times, elimination of the need to measure the dependent variable.

This method can be used to predict various physical properties of asphalt, for example. The software can be used to correlate chemical measurement data such as (but not limited to) near- or mid-infrared (IR) spectroscopy, gel permeation chromatography, asphaltene flocculation titration, ion exchange chromatography, asphaltene solubility subfraction profile analysis, chromatography, or the fully automated saturates, aromatics resins—Asphaltene Determinator™ (SAR-AD) analysis.

The use of this method can be combined with a wide assortment of analytical techniques to provide predictions of many physical properties of oil (petroleum or non-petroleum derived), asphalts, polymers, biological materials or any material for which chemical or spectroscopic measurements can be made. A detailed description of the development of, and certain aspects of, the specialized WRI chemometric software, a key component to this invention, is provided in Glaser et al. report to FHWA (2015) (see Appendix 1), and is incorporated herein by reference. Note that certain aspects of embodiments of the inventive technology, particularly regarding the application of the technology, may be discussed in only rudimentary terms in this Glaser report. Note also that any step of any of the claims can be combined with any step of any of the other claims to describe a particular embodiment of the inventive technology.

Applications for asphalt can include but are not limited to any rheological or empirical mechanical properties, for any type of asphalt binder included modified, roofing, paving, or sealing. More generally, the inventive approach disclosed herein can be used for many other types of materials, and related processes, also. And this approach generates information (e.g., estimate of a dependent variable) that can be used to transform a process relating to any of such materials, or a product that includes any of such materials. A non-exhuastive list of some of these processes (or materials that such processe relate to), and materials, is follows:
Processes relating to durability, as indicated by properties measured at various aging stages, unaged and aged
Product formulation in general
blending proportions based on those properties
designing an additive based on those properties
determining the type and/or amount of additive to be added
Compatibility and phase separation in asphalt binder and consequences in terms of stability, either for asphalt made of blends from refining bases (residues from straight run distillation, solvent deasphalting airblowing, visbreaking, hydrotreating, cracking or coking units), or for any of those blends further modified with any semi-compatible additives, including but not restricted to polymers, acids, waxes, rubbers, amines, and derivatives.
Asphalt and petroleum emulsion ability, storability, breaking, coalescence and curing, and any physical properties of these emulsions and their residues after recovery process.
Asphalt binder and flux aging, short term and long term, w/ w/o UV and moisture (to address both paving and roofing coatings)
Long term durability and performance of highway and roofing materials
Blending properties of asphalts with aged asphalts from recycled paving materials or recycled roofing materials
Asphalt specification parameters
Asphalt binder physical properties, rheological properties in particular, such as complex modulus, phase angle or any combinations or derivatives.
Properties and performance of asphalt binder, asphalt aggregate mixture or chip seals, asphalt shingles or other industrial applications.
Reactivity characteristics of petroleum or petroleum derived fractions or materials for various processes including production, heating, distillation, hydrotreating, coking and others.
Refining an asphalt (or other materi) blend/mix; selecting a bitumen thereofor; modifying a blend recipe; determining an ingredient amount
Fouling characteristics of crude oils in upstream and downstream applications and oil derived materials including fuels and asphalts.
Investigating and predicting properties of polymers, biological materials, biofuels, asphalt binder sealants, asphalt binder rejuvenators
Investigating and predicting properties or effects (whether intended or not) of cosmetics, surfactants, medications and food materials
Hydrocarbon, asphalt, any type of oil, petroleum, coal, and biomass products, fuel, medication, dietary supplements. cosmetics, food, lubricants, and any other materials indicated in this application, or in references incorporated herein.

Additional exemplary applications of embodiments of the inventive software/method may be found, in detail, in: Delfosse, F., et al, Impact of the Bitumen Quality on the Asphalt Mixes Performances, E&E Congress 2016, 6^{th} Eurasphalt & Eurobitume Congress, EE.2016.049 (see Appendix 2); and Glaser, R., et al., Relationships Between Solubility and Chromatographically Defined Bitumen Fractions and Physical Properties, E&E Congress 2016, 6^{th} Eurasphalt & Eurobitume Congress, EE.2016.337 (see Appendix 3), both said papers incorporated herein, by reference, in their entirety.

Again, knowledge obtained from this invention (e.g., estimates of a dependent value) can be used to transform any of such above referenced processes or processes involving any of the above-mentioned materials, in addition to any other process disclosed or indicated herein, or related to any material disclosed or indicated herein. For example, this technology can be used to formulate, blend and mix more cost efficiently hydrocarbons such as long term performing asphalt materials, lubricants, greases, crude oils or any petroleum products, or more generally chemical products, including additives and polymers, making them easier to produce and handle avoiding trial and error based empirical methods. Estimates can be used to transform any of a variety of processes (e.g., modifying a formulation of a hydrocarbon mixture, emulsion, or blend; or a blend or mix of hydrocarbons; or modify a process involving an additives(s) (e.g., designing and selecting better additives), as but a few examples). Where a process is carried out in any manner that, due to information (particularly regarding the independent variable) generated upon use of the inventive software/method, is different from that manner in which the process would be carried out in the absence of such information, said process is said to have been transformed. Similarly, estimates of a dependent variable can be used to transform a product (e.g., one defined or qualified by a dependent variable estimate) from what it would be without such dependent variable information (e.g., as where an estimate of a dependent variable of a certain material/product is used to determine how much or what type of an additive to add to that product to achieve a certain result (e.g., prevent coking)). Transformation of the product or process, in preferred embodiments, results in an improvement, typically to that product or to a product that the process relates to (e.g., a product that the process generates). For example, transformation of an asphalt may lead to an increase in the constituent amounts of one of its ingredients, resulting in an asphalt with improved durability and/or better aging; transformation to an asphalt blending process may lead to a decrease in one ingredient and an increase in another ingredient resulting in an asphalt with better UV resistance. Transformation of a hydrocarbon processing method may involve the use of an additive that would otherwise not be used, or use of an additive in amounts that otherwise would not be observed, to better avoid coking, or allow for higher processing temperatures with confidence that no coking will occur, resulting in more efficient processing.

Note that the inventive technology is not limited to inventive methods, as indeed a system for transforming a process or product may describe, generally, an aspect of the inventive technology. Such system may comprise the following: a linear dependence assignment element that assigns a linear dependence of a dependent variable on “n” number of independent variables; an observation element that yields “p” number of observations to obtain “p” number of measurements for each said dependent variable and said independent variables, wherein “p” is less than the sum of “n”+1; an artificial data generation element that generates artificial data using measurement precision, for at least some of said variables; statistically significant independent variable determiner that determines statistically significant independent variables, wherein said statistically significant independent variables have a statistically significant impact on said dependent variable, and are fewer in number than “n”; a coefficients generator that generates coefficients for each of said statistically significant independent variables; a truncated, closed form mathematical relationship generator that generates a relationship according to which said dependent variable linearly depends from only said statistically significant independent variables, wherein said truncated, closed form mathematical relationship yields results that are sufficiently precise; a dependent variable estimator that uses said relationship, and at least one measurement of each of said at least said statistically significant independent variables to obtain a dependent variable estimate; and a transformation of a process or a product from what said process or said product would be without consideration of said dependent variable estimate. Note that each of said elements may be a subroutine, e.g., a series of encoded instructions, as indicated in the Additional Information section herein. Apparatus/system versions of all method claims filed herewith are disclosed either explicitly herein, or upon consideration of the fact that the disclosure of the steps of generating, determining, producing, developing, truncating, estimating, etc., is deemed disclosure of corollary apparatus steps of a determinator, producer, developer, truncator, estimator, etc., respectively; any and all disclosure particulars that relate to each specific step is also deemed to describe each corollary apparatus componentry.

The WRI chemometric software is especially useful for applications where insufficient observations are available compared to the number of independent measurement variables available. This situation is common in many fields of science and technology, such as spectroscopy, calorimetry, thermogravimetric, chromatography and others.

Additional Information: As can be easily understood from the foregoing, the basic concepts of the present invention may be embodied in a variety of ways. It involves both correlation techniques as well as devices to accomplish the appropriate correlation. In this application, the correlation techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “correlation” should be understood to encompass disclosure of the act of “correlating”—whether explicitly discussed or not—and, conversely, were there effectively disclosure of the act of “correlating”, such a disclosure should be understood to encompass disclosure of a “correlation” and even a “means for correlating.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any acts of law, statutes, regulations, or rules mentioned in this application for patent; or patents, publications, or other references mentioned in this application for patent are hereby incorporated by reference. Any priority case(s) claimed by this application is hereby appended and hereby incorporated by reference. All claims filed herewith are incorporated into this application. Any appendices filed with this application are hereby incorporated into this application. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The International Patent Application or other information list or statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the correlation devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

In addition and as to computer aspects and each aspect amenable to programming or other electronic automation, the applicant(s) should be understood to have support to claim and make a statement of invention to at least: xv) processes performed with the aid of or on a computer as described throughout the above discussion, xvi) a programmable apparatus as described throughout the above discussion, xvii) a computer readable memory encoded with data to direct a computer comprising means or elements which function as described throughout the above discussion, xviii) a computer configured as herein disclosed and described, xix) individual or combined subroutines and programs as herein disclosed and described, xx) a carrier medium carrying computer readable code for control of a computer to carry out separately each and every individual and combined method described herein or in any claim, xxi) a computer program to perform separately each and every individual and combined method disclosed, xxii) a computer program containing all and each combination of means for performing each and every individual and combined step disclosed, xxiii) a storage medium storing each computer program disclosed, xxiv) a signal carrying a computer program disclosed, xxv) the related methods disclosed and described, xxvi) similar, equivalent, and even implicit variations of each of these systems and methods, xxvii) those alternative designs which accomplish each of the functions shown as are disclosed and described, xxviii) those alternative designs and methods which accomplish each of the functions shown as are implicit to accomplish that which is disclosed and described, xxix) each feature, component, and step shown as separate and independent inventions, and xxx) the various combinations and permutations of each of the above.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

### LIST OF REFERENCES TO BE INCORPORATED BY REFERENCE INTO THIS INTERNATIONAL PATENT APPLICATION

### I. US Patents

| | | Kind | Country | Date of | |
|---|---|---|---|---|---|
| Pat. No. | | Code | Code | Issuance | Patentee |
| 1 | 5,590,218 | | US | 1996 Dec. 31 | Ornstein |
| 2 | 5,752,007 | | US | 1998 May 12 | Morrison |
| 3 | 8,370,082 | B2 | US | 2013 Feb. 5 | De Peinder et al. |
| 4 | 4,800,279 | A | US | 1989 Jan. 24 | Indian University |
| | | | | | Foundation |
| 5 | 4,847,793 | | US | 1989 Jul. 11 | Cygnarowicz et al. |

### II. US Publications

| | | Kind | Country | Date of | |
|---|---|---|---|---|---|
| Publication No. | | Code | Code | Issuance | Patentee |
| 1 | 20130067991 | A1 | US | 2013 Mar. 21 | Schabron et al. |

### III. Foreign References

| | | Kind | Country | Date of | |
|---|---|---|---|---|---|
| Pat. No. | | Code | Code | Issuance | Patentee |
| 1 | 2,071,329 | A2 | EP | 2009 Jun. 17 | ENI S.P.A. |
| 2 | 1997014953 | A1 | WO | 1997 Apr. 24 | Shell Int Research |
| 3 | 2,645,695 | A1 | CA | 2009 Jun. 13 | Eni S.P.A. |
| 4 | 1997001096 | A1 | WO | 1997 Jan. 9 | Exxon Research and |
| | | | | | Engineering Company |

### IV. Non Patent Literature

NON PATENT LITERATURE DOCUMENTS

| | |
|---|---|
| 1 | Schabron, J. F., Turner, T. F., Boysen, R. B., Glaser, R. R., Loveridge, J. L., Miknis, F. P. Qin, Q., |
| | Rovani, J. F., Salmans, S. L., 2013, Laboratory and Field Asphalt Binder Aging: |
| | Chemical Changes and Influence on Asphalt Binder Embrittlement; Products: FP 19 |
| | Chemical Changes with Oxidative Aging; FP 20 Linear Amplitude Sweep Test; FP 21 |
| | Application of the Asphaltene Determinator to Asphalt, WRI Report to the Federal |
| | Highway Administration, Contract No. DTFH61-07-D-00005. |
| | 193 pages. |
| 2 | Lima, F. S. G., Leite, L. F. M., 2004, Determination of Asphalt Cement Properties by Near |
| | Infrared Spectroscopy and Chemometrics, Petroleum Science and Technology, 22: 5-6 589-600. |
| | 14 pages. |
| 3 | De Peinder, D. D. Petrauskas, F. Singlenberg, F. Salvatori, T. Visser, F. Soulimani, B. M. |
| | Weckhuysen, 2008, Prediction of Long and Short Residue Properties form Crude Oils form |
| | their Infrared and Near-Infrared Spectroscopy, Applied Spectroscopy, 62(4), 414-422. |
| | 9 pages. |
| 4 | Glaser, R. R., Beemer, A., Turner, T. F., 2015, Chemo-Mechanical Software, Fundamental |
| | Properties of Asphalts and Modified Asphalts III Product: FP 06, Western Research |
| | Institute Report to the Federal Highway Administration, Contract No. DTFH61-07-D- |
| | 00005. |
| | rhttp://www.westernresearch.org/uploadedFiles/Transportation_Technology/FHWA_Research/Fundamental_Properties/ |
| | Technical%20White%20Paper%20FP%2006--Chemo-Mechanical%20Software.pdf |
| | 54 pages. |
| 5 | Schabron, J. F., J. F. Rovani, and M. M. Sanderson, 2010, The Asphaltene Determinator |
| | Method for Automated On-column Precipitation and Re-dissolution of Pericondensed |
| | Asphaltene Components, Energy and Fuels, 24, 5984-5996. |
| 6 | Boysen, R. B., Schabron, J. F., 2013, The Automated Asphaltene Determinator Coupled |
| | with Saturates, Aromatics, Resins Separation for Petroleum Residua Characterization, |
| | Energy Fuels, 27: 4654-4661. |
| 8 | Delfosse, et al. Impact of the bitumen quality on the asphalt mixes performances. E&E |
| | Congress 106. Prague, Czech Republic. Jun. 1-3, 2016. 13 pages. |
| 9 | Glaser et al. Relationships between solubility and chromatographically defined bitumen |
| | fractions and physical properties. E&E Congress 106. Prague, Czech Republic. Jun. 1-3, |
| | 2016. 11 pages. |
| 10 | Wikipedia, “Synthetic Data”. https://en.wikipedia.org/wiki/Synthetic_data. Page last |
| | modified Apr. 1, 2016. |
| 11 | Albuquerque, Georgia et al. “Synthetic Generation of High-Dimensional Datasets”. |
| | http://graphics.tu-bs.de/media/publications/Albuquerque2011SGH.pdf. Posted online 23 |
| | Oct. 2011. 8 pages. |
| 12 | Whiting, Mark, et al. Creating Realistic, Scenario-Based Synthetic Data for Test and |
| | Evaluation of Information Analytics Software. |
| | https://www.purdue.edu/discoverypark/vaccine/assets/pdfs/publications/pdf/Creating%20Realistic,%20Scenario-Based.pdf. |
| | ©2008. 9 pages. |
| 13 | “Dataset generation”. http://www.causality.inf.ethz.ch/data/dataset_generation.html. Date |
| | unknown. |
| 14 | “Artificial Data”. https://www.cs.cmu.edu/afs/cs/project/jair/pub/volume18/thompson03a-html/node19.html. |
| | Cindi Thompson, 2003-01-02. |
| 15 | Brownlee, Jason. 8 Tactics to Combat Imbalanced Classes in Your Machine Learning |
| | Dataset. http://machinelearningmastery.com/tactics-to-combat-imbalanced-classes-in-your-machine-learning-dataset/. |
| | Aug. 19, 2015. 15 pages. |
| 16 | Boysen, Ryan and Schabron, John, Automated HPLC SAR-AD Separation; Fundamental |
| | Properties of Asphalts and Modified Asphalts III Product: FP 01, March 2015 |

What is claimed is:

1. A method for transforming a process or product, comprising the steps of:
assigning linear dependence of a dependent variable on “n” number of independent variables;
performing “p” number of observations to obtain “p” number of measurements for each said dependent variable and said independent variables, wherein “p” is less than the sum of “n”+1;
generating artificial data, using measurement precision, for at least some of said variables;
determining statistically significant independent variables, wherein said statistically significant independent variables have a statistically significant impact on said dependent variable, and are fewer in number than “n”;
generating coefficients for each of said statistically significant independent variables;
developing a truncated, closed form mathematical relationship according to which said dependent variable linearly depends from only said statistically significant independent variables, wherein said truncated, closed form mathematical relationship yields results that are sufficiently precise;
performing at least one observation to obtain at least one measurement of each of at least said statistically significant independent variables;
using said truncated, closed form mathematical relationship, and said at least one measurement of each of said at least said statistically significant independent variables to obtain a dependent variable estimate; and
using said dependent variable estimate to transform a process or a product from what said process or said product would be without consideration of said dependent variable estimate.

2. A method for transforming a process or product as described in claim 1 wherein said observations are made using an IR instrument or a SAR-AD instrument.

3. A method for transforming a process or product as described in claim 1 wherein said step of generating artificial data for at least some of said variables comprises the step of generating artificial data for said dependent variable.

4. A method for transforming a process or product as described in claim 1 wherein said step of generating artificial data for at least some of said variables comprises the step of generating artificial data for a plurality of said independent variables.

5. A method for transforming a process or product as described in claim 4 wherein said step of generating artificial data for at least some of said variables comprises the step of generating artificial data for all “n” of said independent variables.

6. A method for transforming a process or product as described in claim 1 wherein said step of generating artificial data, using measurement precision, for at least some of said variables, comprises the step of generating artificial data using measurement error distribution information.

7. A method for transforming a process or product as described in claim 1 wherein said step of generating artificial data comprises the step of artificially generating enough data so that said linear dependence is mathematically tractable.

8. A method for transforming a process or product as described in claim 7 wherein said step of generating artificial data comprises the step of artificially generating observations such that the total number of observations, actual and artificial, is equal to or greater than said sum of “n”+1.

9. A method for transforming a process or product as described in claim 1 wherein said step of generating artificial data, using measurement precision, for at least some of said variables comprises the steps of delineating, for each said independent variables and said dependent variable, a plurality of ranges centered around a measured variable value; assigning a frequency to each of said ranges according to a known or estimated frequency for each of said ranges; randomly determining a first value within each of said ranges; and generating a plurality of said data for each of said independent variables and said dependent variable according to said frequencies of said ranges for each of said variables.

10. A method for transforming a process or product as described in claim 1 further comprising the step of determining whether any combinations of two or more independent variables have a statistically significant impact on said dependent variable

11. A method for transforming a process or product as described in claim 1 further comprising the step of assessing whether all possible combinations of two independent variables have a statistically significant impact on said dependent variable.

12. A method for transforming a process or product as described in claim 11 further comprising the step of assessing whether all possible combinations of two or more independent variables have a statistically significant impact on said dependent variable.

13. A method for transforming a process or product as described in claim 1 wherein said steps are performed in the order shown.

14. A method for transforming a process or product as described in claim 1 wherein said steps are not performed in the order shown.

15. A method for transforming a process or product as described in claim 1 where at least two of said steps are performed simultaneously.

16. A method for transforming a process or product as described in claim 1 wherein said method is at least partially computer implemented.

17. A method for transforming a process or product as described in claim 16 wherein said steps of generating artificial data, determining statistically significant independent variables, generating coefficients, developing a truncated, closed form mathematical relationship, and using said truncated, closed form mathematical relationship are performed through use of a computer.

18. A method for transforming a process or product as described in claim 1 further comprising the step of preconditioning said measurements for at least some of said independent variables.

19. A method for transforming a process or product as described in claim 18 wherein said step of preconditioning comprises the step of consolidating at least some of said independent variables.

20. A method for transforming a process or product as described in claim 1 further comprising the step of preconditioning said measurements for said dependent variable.

21. A method for transforming a process or product as described in claim 1 further comprising the step of consolidating at least some of said independent variables.

22. A method for transforming a process or product as described in claim 1 further comprising the step of determining whether an acceptably low number of said statistically significant independent variables provide sufficiently precise results when measurements thereof are applied in said truncated, closed form mathematical relationship.

23. A method for transforming a process or product as described in claim 1 further comprising the step of determining whether a minimum precision of results corresponds with an acceptably low number of statistically significant independent variables.

24. A method for transforming a process or product as described in claim 1 wherein said independent variables related to a property selected from the group consisting of: temperature, asphaltene percent, asphaltene fraction percentage, IR wave number/length, UV absorbance, spectroscopy, IR spectroscopy, NIR spectroscopy, MIR band intensities, MIR wavelengths, NMR displacement, spectroscopic peak intensity, UV spectroscopy, RAMAN analysis, SAX analysis, SANS analysis, XRay diffraction, composition, elemental analysis, metal content, microscopy and image analysis property, electronic microscopy, image analysis property, optical microscopy and image analysis property, atomic (AFM) microscopy and image analysis property, tomography, MRI, thermal properties, DSC glass transition temperature, crystallinity, TGA weight loss, HP DSC oxidation induction time, oil component fractions, SAR-AD measured properties, WAX-AD measured properties, SARA fractions, SARA indices, AFT indices, GPC molecular weight, GPC molecular retention times, GPC molecular retention intensities, IEC related property, olefin index, acidity-basicity property, TAN, TBN, elemental analysis property, microscopy and image analysis property, electronic microscopy and image analysis, optical microscopy and image analysis, atomic (AFM) microscopy and image analysis, tomographic property, and MRI.

25. A method for transforming a process or product as described in claim 1 wherein said step of performing “p” number of observations is accomplished at least in part through the use of a method or instrument selected from the group consisting of: IR spectrometer, NIR spectrometer, MIR spectrometer, SAR-AD analyzer, WAD analyzer, any SARA method, DSR, BBR, ABCD, DMA, mechanical test, fouling analyzer, NMR (1H and 13C), GPC/SEC, DSC, IEC and AFT.

26. A method for transforming a process or product as described in claim 1 wherein said step of performing “p” number of observations comprises the step of performing observations of a material selected from the group consisting of: petroleum product, coal product, hydrocarbonaceous material, biomass product, asphalt, bitumen, fuel, medication, dietary supplements, cosmetics, food, and lubricant.

27. A method for transforming a process or product as described in claim 1 wherein said dependent variables relate to a property, phenomonen or parameter selected from the group consisting of crude oil property, petroleum fouling parameter, coking, emulsion ability, stability, emulsion instability, gas/fuel cetane number, gas/fuel octane numbers, lubricant property, anti-wear property, viscosity index, oxidation resistance, fluidity, tribology, asphalt penetration, ring and ball softening point, fraass brittle point, viscosity, modulus, phase angle, superpave properties, DSR, BBR critical temperatures, oxidation resistance short term and long term, material fatigue resistance, brittleness, product formulation, hardness, elasticity, plasticity, deformation, roughness, density, organic or inorganic material oxidation, material weatherability, material durability, material inflammability, explosiveness, carcinogenicity, mutagenicity, metal corrosion, liquid or paste fluidity, thixotropy, viscosity, material density, perfume smell, spraying ability, medication efficiency/effectiveness, product formula, product formulation, fluid viscosity, material hardness and material reflectivity.

28. A method for transforming a process or product as described in claim 1 wherein said step of using said dependent variable estimate to transform a process or product comprises the step of using said dependent variable estimate to transform a process or product selected from the group consisting of: processes relating to durability measured at various aged and unaged aging stages, blending process, blending proportions, blending proportions based on durability, product formulation, additive design, additive amount for addition to hydrocarbon or other product, additive type for addition to hydrocarbon or other product, compatibility and phase separation in asphalt binder and consequences in terms of stability, either for asphalt made of blends from refining bases (residues from straight run distillation, solvent deasphalting airblowing, visbreaking, hydrotreating, cracking or coking units), or for any of those blends further modified with any semi-compatible additives, including but not restricted to polymers, acids, waxes, rubbers, amines, and derivatives, asphalt and petroleum emulsion ability, storability, breaking, coalescence and curing, and any physical properties of these emulsions and their residues after recovery process, asphalt binder and flux aging, short term and long term, with and without UV and moisture (to address both paving and roofing coatings), long term durability and performance of highway and roofing materials, blending properties of asphalts with aged asphalts from recycled paving materials or recycled roofing materials, product formulation, asphalt specification parameters, asphalt binder physical properties, rheological properties in particular, such as complex modulus, phase angle or any combinations or derivatives, properties and performance of asphalt binder, asphalt aggregate mixture or chip seals, asphalt shingles or other industrial applications, reactivity characteristics of petroleum or petroleum derived fractions or materials for various processes including production, heating, distillation, hydrotreating, coking and others, refining an asphalt (or other material) blend/mix; selecting a bitumen therefor; modifying a blend recipe; determining an ingredient amount, fouling characteristics of crude oils in upstream and downstream applications and oil derived materials including fuels and asphalts, investigating and predicting properties of polymers, biological materials, biofuels, asphalt binder sealants, asphalt binder rejuvenators, investigating and predicting properties or effects (whether intended or not) of cosmetics, surfactants, medications and food materials, hydrocarbon, asphalt, any type of oil, petroleum, coal, and biomass products, fuel, medication, dietary supplements, cosmetics, food, lubricants.

29. A method of transforming a product or process comprising the steps of:
performing at least one observation to obtain measured data that includes at least one measurement of each of independent and dependent variables;
generating artificial data, using measurement precision, for at least some of said variables, said step of generating artificial data comprising the steps of
delineating, for each of at least some of said independent variables, and for said dependent variable, a plurality of ranges centered around a measured variable value;
assigning a frequency to each of said ranges according to a known or estimated frequency for each of said ranges;
randomly determining a first value within each of said ranges; and
generating a plurality of said artificial data for each of said at least some of said independent variables and said dependent variable according to said frequencies of said ranges for each of said at least some of said variables;
said method further comprising the steps of:
using said artificial data and said measured data to determine coefficients of a linear relationship between said at least some of said independent variables and said dependent variable, thereby determining a closed form mathematical relationship between said at least some of said independent variables and said dependent variable;
performing at least one observation to obtain at least one measurement of said each of said at least some of said independent variables;
using said closed form mathematical relationship, and said at least one measurement of each of at least some of said independent variables, to obtain a dependent variable estimate; and
using said dependent variable estimate to transform a process or a product from what said process or said product would be without consideration of said dependent variable estimate.

30. A method of transforming a product or process as described in claim 29 further comprising the step of determining statistically significant independent variables, wherein said statistically significant independent variables have a statistically significant impact on said dependent variable.

31. A method of transforming a product or process as described in claim 30 wherein said at least some of said independent variables comprises said statistically significant independent variables.

32. A method of transforming a product or process as described in claim 29 further comprising the step of consolidating at least some of said independent variables.

33. A method of transforming a product or process as described in claim 29 wherein said step of generating artificial data for at least some of said variables comprises the step of generating artificial data for all “n” of said independent variables.

34. A method of transforming a product or process as described in claim 29 wherein said step of generating artificial data comprises the step of artificially generating enough data so that said linear dependence is mathematically tractable.

35. A method of transforming a product or process as described in claim 29 wherein said steps are performed in the order shown.

36. A method of transforming a product or process as described in claim 29 wherein said steps are not performed in the order shown.

37. A method of transforming a product or process as described in claim 29 wherein at least two of said steps are performed simultaneously.

38. A method of transforming a product or process as described in claim 29 wherein said method is at least partially computer implemented.

39. A method of transforming a product or process as described in claim 29 further comprising the step of preconditioning said measurement data for said at least some of said independent variables.

40. A method of transforming a product or process as described in claim 39 wherein said step of preconditioning comprises the step of consolidating at least some of said independent variables.

41. A method of transforming a product or process as described in claim 29 further comprising the step of preconditioning said measurements for said dependent variable.

42. A method of transforming a product or process as described in claim 29 wherein said independent variables related to a property selected from the group consisting of: temperature, asphaltene percent, asphaltene fraction percentage, IR wave number/length, UV absorbance, spectroscopy, IR spectroscopy, NIR spectroscopy, MIR band intensities, MIR wavelength, NMR displacement, spectroscopic peak intensity, UV spectroscopy, RAMAN analysis, SAX analysis, SANS analysis, XRay diffraction, elemental analysis, metal content, microscopy and image analysis property, electronic microscopy, image analysis property, optical microscopy and image analysis property, atomic (AFM) microscopy and image analysis property, tomography, MRI, thermal properties, DSC glass transition temperature, crystallinity, TGA weight loss, HP DSC oxidation induction time, oil component fractions, SAR-AD measured properties, WAX-AD measured properties, SARA fractions, SARA indices, AFT indices, GPC molecular weight, GPC molecular retention times, GPC molecular retention intensities, IEC related property, olefin index, acidity-basicity property, TAN, TBN, elemental analysis property, microscopy and image analysis property, electronic microscopy and image analysis, optical microscopy and image analysis, atomic (AFM) microscopy and image analysis, tomographic property, and MRI.

43. A method of transforming a product or process as described in claim 29 wherein said step of performing at least one observation is accomplished at least in part through the use of a method or instrument selected from the group consisting of: IR spectrometer, NIR spectrometer, MIR spectrometer, SAR-AD analyzer, WAD analyzer, any SARA method, DSR, BBR, ABCD, DMA, mechanical test, fouling analyzer, NMR (1H and 13C), GPC/SEC, DSC, IEC and AFT.

44. A method of transforming a product or process as described in claim 29 wherein said step of performing at least one observation comprises the step of performing at least one observation of a material selected from the group consisting of: petroleum product, coal product, hydrocarbonaceous material, biomass product, asphalt, bitumen, fuel, medication, dietary supplements, cosmetics, food, and lubricant.

45. A method of transforming a product or process as described in claim 29 wherein said dependent variables relate to a property, phenomonen or parameter selected from the group consisting of crude oil property, petroleum fouling parameter, coking, emulsion ability, stability, emulsion instability, gas/fuel cetane number, gas/fuel octane numbers, lubricant property, anti-wear property, viscosity index, oxidation resistance, fluidity, tribology, product formula, product formulation, asphalt penetration, ring and ball softening point, fraass brittle point, viscosity, modulus, phase angle, superpave properties, DSR, BBR critical temperatures, oxidation resistance short term and long term, material fatigue resistance, brittleness, hardness, elasticity, plasticity, deformation, roughness, density, organic or inorganic material oxidation, material weatherability, material durability, material inflammability, explosiveness, carcinogenicity, mutagenicity, metal corrosion, liquid or paste fluidity, thixotropy, viscosity, material density, perfume smell, spraying ability, medication efficiency/effectiveness, fluid viscosity, material hardness and material reflectivity.

46. A method of transforming a product or process as described in claim 29 wherein said step of using said dependent variable estimate to transform a process or product comprises the step of using said dependent variable estimate to transform a process or product selected from the group consisting of: processes relating to durability measured at various aged and unaged aging stages, blending process, blending proportions, blending proportions based on durability, additive design, additive amount for addition to hydrocarbon or other product, additive type for addition to hydrocarbon or other product, compatibility and phase separation in asphalt binder and consequences in terms of stability, either for asphalt made of blends from refining bases (residues from straight run distillation, solvent deasphalting airblowing, visbreaking, hydrotreating, cracking or coking units), or for any of those blends further modified with any semi-compatible additives, including but not restricted to polymers, acids, waxes, rubbers, amines, and derivatives, asphalt and petroleum emulsion ability, storability, breaking, coalescence and curing, and any physical properties of these emulsions and their residues after recovery process, asphalt binder and flux aging, short term and long term, with and without UV and moisture (to address both paving and roofing coatings), long term durability and performance of highway and roofing materials, blending properties of asphalts with aged asphalts from recycled paving materials or recycled roofing materials, product formulation, asphalt specification parameters, asphalt binder physical properties, rheological properties in particular, such as complex modulus, phase angle or any combinations or derivatives, properties and performance of asphalt binder, asphalt aggregate mixture or chip seals, asphalt shingles or other industrial applications, reactivity characteristics of petroleum or petroleum derived fractions or materials for various processes including production, heating, distillation, hydrotreating, coking and others, refining an asphalt (or other material) blend/mix; selecting a bitumen therefor; modifying a blend recipe; determining an ingredient amount, fouling characteristics of crude oils in upstream and downstream applications and oil derived materials including fuels and asphalts, investigating and predicting properties of polymers, biological materials, biofuels, asphalt binder sealants, asphalt binder rejuvenators, investigating and predicting properties or effects (whether intended or not) of cosmetics, surfactants, medications and food materials, hydrocarbon, asphalt, any type of oil, petroleum, coal, and biomass products, fuel, medication, dietary supplements, cosmetics, food, lubricants.

47. A system for transforming a process or product, comprising the steps of:
a linear dependence assignment element that assigns a linear dependence of a dependent variable on “n” number of independent variables;
an observation element that yields “p” number of observations to obtain “p” number of measurements for each said dependent variable and said independent variables, wherein “p” is less than the sum of “n”+1;
an artificial data generation element that generates artificial data using measurement precision, for at least some of said variables;
statistically significant independent variable determiner that determines statistically significant independent variables, wherein said statistically significant independent variables have a statistically significant impact on said dependent variable, and are fewer in number than “n”;
a coefficients generator that generates coefficients for each of said statistically significant independent variables;
a truncated, closed form mathematical relationship generator that generates a relationship according to which said dependent variable linearly depends from only said statistically significant independent variables, wherein said truncated, closed form mathematical relationship yields results that are sufficiently precise;
a dependent variable estimator that uses said relationship, and at least one measurement of each of said at least said statistically significant independent variables to obtain a dependent variable estimate; and
a transformation of a process or a product from what said process or said product would be without consideration of said dependent variable estimate.
